SEQUENCE LISTING (3 sequences)

SEQ ID NO: 1 - protein, 9 aa, Artificial Sequence
  peptide that promotes complement activation
CLSAHHHMC

SEQ ID NO: 2 - protein, 9 aa, Artificial Sequence
  peptide that promotes complement activation
CPSSPPHMC

SEQ ID NO: 3 - protein, 9 aa, Artificial Sequence
  peptide that promotes complement activation
CPGKASPWC

(19) United States
(12) Patent Application Publication
(10) Pub. No.: US 2005/0090447 A1
(43) Pub. Date: Apr. 28, 2005

(54) Peptides that promote complement activation

(75) Inventors: Richard J. Johnson, Mundelein, IL (US); Shelley A. Maves, Mokena, IL (US)

Correspondence Address: BAXTER HEALTHCARE CORPORATION, ONE BAXTER PARKWAY, DF2-2E, DEERFIELD, IL, 60015, US

(73) Assignees: Baxter International Inc.; Baxter Healthcare S.A.

(21) Appl. No.: 10/938,088

(22) Filed: Sep. 10, 2004

Related U.S. Application Data
(60) Provisional application No. 60/501,704, filed on Sep. 10, 2003.

(51) Int. Cl. A61K 38/08; C07K 7/06

(52) U.S. Cl. 514/15; 530/328

(57) ABSTRACT

The present invention relates to compositions, including pharmaceutical compositions that promote complement activation, and contain a polypeptide X₁-C-X₂-X₃-X₄-X₅-X₆-X₇-X₈-C-X₉ as described. The invention further relates to a method of promoting complement activation in a patient by administering a pharmaceutical composition as described herein. Further provided are a wound dressing and an anti-tumor cell antigen antibody formulation containing pharmaceutical compositions of the present invention.

### FIELD OF THE INVENTION

The present invention relates to a composition that promotes complement activation. The invention further relates to a method of promoting complement activation in a patient by administering to the patient a pharmaceutical composition that promotes complement activation. Further provided are a wound dressing and anti-tumor cell antibody formulation that contain the pharmaceutical compositions described herein.

### BACKGROUND OF THE INVENTION

The human immune system is equipped with several defense mechanisms to respond to bacterial, viral, or parasitic infection and injury. One of such defense mechanisms is the complement system, which plays a role both in innate and acquired immunity (see e.g. Cooper (1985), Adv. Immunol. 61:201-283; Liszewski et al. (1996), Adv. In Immunol. 61:201-282; Matsushita (1996), Microbiol. Immunol. 40:887-893; Sengelov (1995), Critical Review in Immunol. 15:107-131). The complement system directly and indirectly contributes to both innate inflammatory reactions as well as cellular (i.e. adaptive) immune responses. This array of effector functions is due to the activity of a number of complement components and their receptors on various cells. One of the principal functions of complement is to serve as a primitive self-nonself discriminatory defense system. This is accomplished by coating a foreign material with complement fragments and recruiting phagocytic cells that attempt to destroy and digest the “intruder”.

Complement refers to a group of plasma proteins that are known to be necessary for antibody-mediated bactericidal activity. The complement system is composed of more than 30 distinct plasma and membrane bound proteins involving three separate pathways: classical, alternative and the lectin pathway. The C3 protein sits at the juncture of the classical and alternative pathways and represents one of the critical control points. Cleavage of C3 yields C3a and C3b. C3b molecules then react with a site on the C4b protein, creating a C3b-C4b-C2b complex that acts as a C5 convertase. Proteolytic activation of C5 occurs only after it is bound to the C3b portion of the C5 convertase on the surface of an activator (e.g., the immune complex). Like C3, C5 is also cleaved by C2b to produce fragments designated C5a (16,000 Da) and C5b (170,000 Da). The C5b molecule combines with the proteins of the terminal components to form the membrane attack complex described below. C5a is a potent inflammatory mediator and is responsible for many of the adverse reactions normally attributed to complement activation in various clinical settings.

The classical pathway (CP) of complement activation is activated primarily by immune complexes (ICs), but also by other proteins such as C-Reactive Protein, Serum Amyloid Protein, amyloid fibrils, and apoptotic bodies (Cooper, 1985).

The lectin pathway, discovered in the 1990s (Matsushita, 1996) is composed of lectins like mannose binding protein (or mannan binding lectin, MBL) and two MBL-associated serine proteases (MASP-1 and MASP-2) (see Wong et al, 1999). Upon activation of MBL-MASP-1-MASP-2, the MASP protease components cleave C4 and C2 forming a CP C3 convertase described above.

In the alternative pathway (AP) of complement activation, C3 is cleaved to form C3b in a mostly hydrolyzed and inactivated form. This process has been termed “C3 tickover,” a continuous and spontaneous process that ensures that whenever an activating surface (a bacterium, biomaterial, etc) presents itself, reactive C3b molecules will be available to mark the surface as foreign. Eventually, a C3b molecule attaches to one of the C3 convertase sites by direct attachment to the C3b protein component of the enzyme. This C3b-C3b-Bb complex is the alternative pathway C5 convertase and, in a manner reminiscent of the CP C5 convertase, converts C5 to C5b and C5a.

All three pathways lead to a common point: cleavage of C5 to produce C5b and C5a. C5a is a potent inflammatory mediator. The production of C5b initiates the formation of a macromolecular complex of proteins called the membrane attack complex (MAC) that disrupts the cellular lipid bilayer, leading to cell death. Even at sublytic levels, formation of MAC on host cells results in a number of activation responses (elevated Ca+2, arachadonic acid metabolism, cytokine production).

Deficiency of a number of complement components has been linked with autoimmune diseases and inability to respond to pathogens properly. In particular, there is a strong association between immune complex diseases and the deficiencies of early components C1, C4, and C2 of the classical pathway. Approximately 90% of C1- and C4-deficient patients are afflicted with systemic lupus erythematosus (SLE). Furthermore, approximately 50% of individuals who are C2-deficient develop SLE or a related illness. There is also an increased frequency of infection in some of these patients.

In individuals with homozygous C3 deficiency, pyogenic infections with encapsulated bacteria are severe, recurrent, and can be life-threatening. Excessive infections with gram-negative bacteria, particularly Neisseria are seen in individuals with homozygous deficiency of the late components (C5, C6, C7, or C8) or of components D and P of the alternative pathway.

Accordingly, administration of complement components to individuals who lack them is a useful therapy for patients who lack these components. In addition, complement activation may be desirable in cases where patients are immuno-compromised, such as undergoing surgery or suffering from burns, which could make them more susceptible to infections. Hence, compositions capable of promoting complement activation would be useful in such applications and in a number of other clinical cases.

### SUMMARY OF THE INVENTION

In one embodiment, the present invention relates to a composition for promoting complement activation, wherein the composition comprises a polypeptide comprising a sequence X₁-C-X₂-X₃-X₄-X₅-X₆-X₇-X₈-
C-X₉, wherein
C is cysteine;
X₁ is a hydrogen atom, an amino acid residue, or a bond covalently linking the polypeptide to another component of the composition;
X₂ is a neutral non-polar amino acid residue;
X₃ is a neutral polar amino acid residue;
X₄, X₅, X₆, and X₇ are independently any amino acid residue;
X₈ is a neutral non-polar amino acid residue; and
X₉ is a hydrogen atom, an amino acid residue, or a bond covalently linking the polypeptide to another component of the composition.

In another embodiment, the present invention relates to a composition for promoting complement activation, wherein said composition comprises a polypeptide comprising a sequence X₁-C-X₂-X₃-X₄-X₅-X₆-X₇-X₈-C-X₉, wherein
C is cysteine;
X₁ is an amino acid residue;
X₂ is a neutral non-polar amino acid residue;
X₃ is a neutral polar amino acid residue;
X₄, X₅, X₆, and X₇ are independently any amino acid residue;
X₈ is a neutral non-polar amino acid residue; and
X₉ is an amino acid residue.

Further provided is a pharmaceutical composition comprising a polypeptide comprising a sequence X₁-C-X₂-X₃-X₄-X₅-X₆-X₇-X₈-C-X₉, wherein
C is cysteine;
X₁ is a hydrogen atom, an amino acid residue, or a bond covalently linking the polypeptide to another component of the composition;
X₂ is a neutral non-polar amino acid residue;
X₃ is a neutral polar amino acid residue;
X₄, X₅, X₆, and X₇ are independently any amino acid residue;
X₈ is a neutral non-polar amino acid residue; and
X₉ is a hydrogen atom, an amino acid residue, or a bond covalently linking the polypeptide to another component of the composition.

In another embodiment provided is a pharmaceutical composition comprising a polypeptide comprising a sequence X₁-C-X₂-X₃-X₄-X₅-X₆-X₇-X₈-C-X₉, wherein
C is cysteine;
X₁ is an amino acid residue;
X₂ is a neutral non-polar amino acid residue;
X₃ is a neutral polar amino acid residue;
X₄, X₅, X₆, and X₇ are independently any amino acid residue;
X₈ is a neutral non-polar amino acid residue; and
X₉ is an amino acid residue.

Another embodiment of the present invention is a method for promoting complement activation in a patient by administering to the patient a therapeutically effective amount of the pharmaceutical composition described herein.

Further provided are a wound dressing and an anti-tumor cell antigen antibody formulation, which comprise therapeutically effective amounts of the pharmaceutical compositions described herein.

Other objects and features will be in part apparent and in part pointed out hereinafter.

### BRIEF DESCRIPTION OF THE FIGURES

FIG. 1 is a bar graph depicting the binding of peptide #1, peptide #2, and peptide #3 to C5. Mean absorbance at 490 nm versus the amount of phage added (pfu) is plotted for determining binding of phage-displayed peptides #1, 2, and 3 to biotinylated C5 by monitoring the phage bound with an anti-M13 antibody. Wild-type M13 phage without a peptide fused to the pill protein was used as a negative control for this assay.

FIGS. 2A and 2B are bar graphs depicting percent hemolysis above control versus peptide concentration with peptides 1, 2, and 3 for classical pathway activation (FIG. 2A), and alternative pathway activation (FIG. 2B). Percent hemolysis above control is the percent lysis of erythrocytes for each peptide concentration minus the lysis of erythrocytes in the absence of peptide.

FIGS. 3A and 3B are bar graphs depicting the level of complement activation in the presence of peptides (1, 2, or 3) with zymosan activated plasma. FIG. 3A depicts C5a production in ng/ml for increasing concentrations of peptides 1, 2, and 3 and no peptide in the presence of 10 mg/ml of zymosan as a complement activator. FIG. 3B depicts C3a concentration for increasing concentrations of peptides in the presence of 0.5 mg/ml of zymosan.

FIG. 4 is a bar graph depicting bacteriocidal activity of peptides against E. coli O7:K1:NM. Controls were run with plasma alone, complement inactivated (heat inactivated) plasma, and PBS/0.5% BSA.

### ABBREVIATIONS AND DEFINITIONS

To facilitate understanding of the invention, a number of terms are defined below:

The term “analog” as used herein refers to a molecule substantially similar in function to either the entire molecule or to a fragment thereof. An analog may contain chemical moieties that are not normally a part of the molecule, but that may, for example, improve the molecule's half-life or decrease its toxicity. Moieties capable of mediating such effects are disclosed in Remington's Pharmaceutical Sciences (1980).

As used herein, the term “amino acid” is used in its broadest sense, and includes naturally occurring amino acids as well as non-naturally occurring amino acids, including amino acid analogs and derivatives. The latter includes molecules containing an amino acid moiety. One skilled in the art will recognize, in view of this broad definition, that reference herein to an amino acid includes, for example, naturally occurring proteogenic L-amino acids; D-amino acids; chemically modified amino acids such as amino acid analogs and derivatives; naturally occurring non-proteogenic amino acids; and chemically synthesized compounds having properties known in the art to be characteristic of amino acids.

As used herein, “bactericidal” refers to the ability to kill bacteria.

“BSA” is an abbreviation for bovine serum albumin.

As used herein, “complement-mediated lysis,” “complement-dependent lysis,” “complement-mediated cytotoxicity,” or “complement-dependent cytotoxicity” all generally mean the process by which the complement cascade is activated, multi-component complexes are assembled, ultimately generating a lytic complex that has direct lytic action, causing cell permeabilization. Therapeutic agent-targeting agents for use in inducing complement-mediated lysis will generally include an antibody Fc portion.

The term “hydrophobic” when used in reference to amino acids refers to those amino acids which have nonpolar side chains. Hydrophobic amino acids include valine, leucine, isoleucine, cysteine and methionine. Three hydrophobic amino acids have aromatic side chains. Accordingly, the term “aromatic” when used in reference to amino acids refers to the three aromatic hydrophobic amino acids phenylalanine, tyrosine and tryptophan.

The term “parenteral” as used herein refers to an administration which is not through the alimentary canal but rather by injection through some other route, as subcutaneous, intramuscular, intraorbital, intracapsular, intraspinal, intrasternal, intravenous, etc. Formulation of drugs is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

“PBS” is an abbreviation for phosphate buffered saline.

The term “pharmaceutically acceptable” is used adjectivally herein to mean that the modified noun is appropriate for use in a pharmaceutical product; that is the pharmaceutically acceptable material is relatively safe and/or non-toxic, though not necessarily providing a separable therapeutic benefit by itself.

As used herein, “polynucleotide” and “oligonucleotide” are used interchangeably and mean a polymer of at least 2 nucleotides joined together by phosphodiester bond(s) and may consist of either ribonucleotides or deoxyribonucleotides.

The term “polypeptide” when used herein refers to two or more amino acids that are linked by peptide bond(s), regardless of length, functionality, environment, or associated molecule(s). Typically, the polypeptide is at least 4 amino acid residues in length and can range up to a full-length protein. As used herein, “polypeptide,” “peptide,” and “protein” are used interchangeably.

“RBC” is an abbreviation for red blood cells.

As used herein, “sequence” means the linear order in which monomers occur in a polymer, for example, the order of amino acids in a polypeptide or the order of nucleotides in a polynucleotide.

The term “subject” for purposes of treatment includes any human or animal subject in need of complement activation. The subject can be a domestic livestock species, a laboratory animal species, a zoo animal or a companion animal. In one embodiment, the subject is a mammal. In another embodiment, the mammal is a human being. The terms “subject” and “patient” are used interchangeably herein.

The phrase “therapeutically-effective” is intended to qualify the amount of an agent or combination of two or more agents, which will achieve the goal of improvement in disorder severity and the frequency of incidence over no treatment.

The term “treatment” includes alleviation, elimination of causation of or prevention of undesirable symptoms associated with a disease or disorder. Treatment as used herein includes prophylactic treatment.

The term “variant” as used herein refers to a molecule substantially similar in structure and biological activity or immunological properties to either the entire molecule or a fragment thereof. Thus, provided that two molecules possess a similar activity, they are considered variants even if the sequence of their amino acid residues is not identical.

### DETAILED DESCRIPTION OF THE INVENTION

The present invention relates to compositions and methods that promote complement activation, and uses thereof.

In one embodiment, the present invention provides a composition comprising a polypeptide X₁-C-X₂-X₃-X₄-X₅-X₆-X₇-X₈-C-X₉, wherein
C is cysteine;
X₁ is a hydrogen atom, an amino acid residue, or a bond covalently linking the polypeptide to another component of the composition;
X₂ is a neutral non-polar amino acid residue;
X₃ is a neutral polar amino acid residue;
X₄, X₅, X₆, and X₇ are independently any amino acid residue;
X₈ is a neutral non-polar amino acid residue; and
X₉ is a hydrogen atom, an amino acid residue, or a bond covalently linking the polypeptide to another component of the composition.

Neutral non-polar amino acids include alanine, leucine, isoleucine, valine, proline, phenylalanine, tryptophan, and methionine, and neutral polar amino acids include glycine, serine, threonine, cysteine, tyrosine, asparagine, and glutamine.

X₁ can be a hydrogen atom, an amino acid residue, or a covalent bond linking the polypeptide to another component of the composition. In one embodiment, X₁ is a hydrogen atom that is attached to the terminal amino group of the cysteine. In this embodiment, the amino-terminus of the polypeptide sequence of the composition is the cysteine.

In another embodiment, X₁ is an amino acid residue. This amino acid residue can be selected from any of the naturally occurring amino acids such as proteogenic L-amino acids (i.e., the 20 amino acids normally incorporated into proteins) as well as D-amino acids and non-proteogenic amino acids. Non-proteogenic amino acids are generally metabolites or analogues of the proteogenic amino acids. Non-limiting examples of naturally occurring non-proteogenic amino acids include ornithine, taurine, hydroxyproline, hydroxylysine, norleucine, β-alanine, gamma amino butyric acid, selenocysteine, phosphoserine, pyroglutamic acid, and pyrrolysine. The _{X1} amino acid may also be selected from non-naturally occurring amino acids. Non-naturally occurring amino acids include, but are not limited to, amino acid derivatives and analogs. Non-limiting examples of amino acid derivates include selenomethionine, telluromethionine, and p-aminophenylalanine, fluorinated amino acids (e.g., fluorinated tryptophan, tyrosine and phenylalanine), nitrophenylalanine, nitrobenzoxadiazolyl-L-lysine, deoxymethylarginine, and cyclohexylalanine. Amino acid analogs include chemically synthesized compounds having properties known in the art to be characteristic of amino acids, examples of which include, but are not limited to, the tryptophan “analog” b-selenolo[3,2-b]pyrrolylalanine and the proline “analog” thiaproline (1,3-thiazolidine-4-carboxylic acid).

Similarly to X₁, X₉ can be a hydrogen atom, an amino acid residue, or a bond covalently linking the polypeptide to another component of the composition. In certain embodiments, wherein X₉ is a hydrogen atom, it is attached to the C-terminal cysteine, making this cysteine the C-terminus of the amino acid sequence. In embodiments wherein X₉ is an amino acid residue or a bond covalently linking it to another component in the composition, X₉ can be selected from the same amino acid residues and bonds that were described above for X₁. By way of example, the peptides containing the amino acid sequences C-X₂-X₃-X₄-X₅-X₆-X₇-X₈-C were modified to include N-terminal tyrosine (X₁) and three C-terminal glycines (X₉) to mimic the linkage to the plll fusion protein.

In yet another embodiment, when X₁ and/or X₉ are bonds covalently linking the polypeptide C-X₂-X₃-X₄-X₅-X₆-X₇-X₈-C (“C-X₂- . . . X₈-C”) to another component, a number of different biological molecules can be created. Such components may include, but are not limited to vitamins, proteins, polypeptides, carbohydrates, polysaccharides, lipids, lipopolysaccharides, nucleic acids, and biomaterials. These components will have a multiplicity of sites to which the peptides can be coupled. Hence, the biological molecule includes one or more components covalently coupled together, e.g., a nucleic acid coupled to a peptide, either directly or through a linker. For example, a biological molecule may be selected from, e.g., protein-C-X₂- . . . X₈-C; protein-C-X₂- . . . X₈-C-protein; nucleic acid-C-X₂- . . . X₈-C-nucleic acid; C-X₂- . . . X₈-C-lipid; protein-C-X₂- . . . X₈-C-lipid; vitamin-C-X₂- . . . X₈-C, and a number of other combinations. Suitable vitamins include, but are not limited to, biotin. Vitamins, such as biotin are known to promote delivery of agents into the blood. Furthermore, biotin/avidin systems are well known in the art. See, e.g., Wilcheck and Bayer (1990), Methods of Enzymology 184 (Academic Press). Suitable proteins include, but are not limited to, albumins (e.g., bovine serum albumin, ovalbumin, human serum albumin), immunoglobulins, thyroglobulins (e.g., bovine thyroglobulin), and hemocyanins (e.g., Keyhole Limpet hemocyanin). Suitable polypeptides include, but are not limited to, polylysine and polyalaninelysine. Suitable polysaccharides include, but are not limited to, dextrans of various sizes (e.g., 12,000 to 500,000 molecular weight). Suitable biomaterials include, but are not limited to, various artificial implants, pacemakers, valves, catheters, and membranes (e.g. dialyzer), as well as synthetic polymers such as polypropylene oxide (PPO) and polyethylene glycol (PEG).

Components coupled to the polypeptide of the composition may play a role in a variety of functions well known in the art. For example, components could include fusion constructs used for targeted delivery of complement inhibitors (see e.g. Song et al (2003), J. Clinical Investigation, 111(12):1875-85; Zhang et al. (2001), J. Biol. Chem. 276(29):27290-95). Targeting could also occur through fusion of the composition with another peptide (see e.g. Cancer Research, 57:1442-1446 (1997)). In a further example, when a biological molecule is, e.g. protein-C-X₂- . . . X₈-C, a protein may be an antibody or a fragment thereof specific for a type of cells, thereby allowing for the targeting of C-X₂- . . . X₈-C to that type of cells. Furthermore, coupling that is performed to increase the size of the biological molecule may be useful as larger molecules tend to have a longer plasma half-life. By way of example, components such as PEG (through pegylation of the polypeptide) can extend the in vivo half-life of complement inhibitor compositions (see e.g. Wang (2002), Advanced Drug Deliv. Reviews, 54:547-570). In yet another non-limiting example of coupling function, glycosylation (i.e. coupling the polypeptide to certain carbohydrates) can improve intestinal absorption of the polypeptide-containing composition of the invention (see e.g. J. Pharmaceutical Sciences, 87(3):326-332 (1998)).

The polypeptides of this composition may be covalently coupled to other components of the composition using methods and agents well known in the art. Suitable agents include glutaraldehyde, carbodiimide, cyanoborohydride, diimidoesters, periodate, alkylhalides, succinimides, dimethylpimelimidate and dimaleimides (see Blait and Ghose (1983), J. Immunol. Methods 59:129; Blair and Ghose, (1981) Cancer Res. 41:2700; Gauthier et al. (1982), J. Exp. Med. 156:766-777). For a list of possible coupling agents, see generally Catalog And Handbook (1994-1995) and Products Catalog (1997), Pierce Chemical Co., Rockford, Ill. Additional references concerning carriers and techniques for coupling polypeptides thereto are: Erlanger (1980), Methods In Enzymology 70:85-104; Makela and Seppala (1986), Handbook of Experimental Immunology (Blackwell); Parker (1976), Radioimmunoassay of Biologically Active Compounds (Prentice-Hall); Butler (1974), J. Immunol. Meth., 7:1-24; Weinryb and Shroff (1979), Drug. Metab. Rev. 10:271-83; Broughton and Strong (1976), Clin. Chem. 22:726-32; Playfair et al. (1974), Br. Med. Bull. 30:24-31.

In one embodiment of the present invention, X₂ is a neutral non-polar amino acid residue. In this embodiment, X₂ may be selected from alanine, leucine, isoleucine, valine, proline, phenylalanine, tryptophan, methionine, and analogs and derivatives thereof, as discussed previously. In another embodiment, X₂ is leucine or proline.

X₃ is a neutral polar amino acid as discussed above. In one embodiment, X₃ is selected from serine or glycine.

X₄, X₅, X₆, and X₇ are independently an amino acid residue, and can be selected from naturally occurring amino acids and non-proteogenic amino acids as described above.

X₈ is a neutral non-polar amino acid. In one embodiment, X₈ is methionine or tryptophan.

In one embodiment, the composition includes a polypeptide X₁-C-X₂-X₃-X₄-X₅-X₆-X₇-X₈-C-X₉, wherein C is cysteine, X₁, X₄, X₅, X₆, X₇, and X₉ are independently an amino acid residue, X₂ is leucine or proline, X₃ is a neutral polar amino acid, and X₈ is a neutral non-polar amino acid.

In another embodiment, the composition includes a polypeptide X₁-C-X₂-X₃-X₄-X₅-X₆-X₇-X₈-C-X₉, wherein C is cysteine, X₁, X₄, X₅, X₆, X₇, and X₉ are independently an amino acid residue, X₂ is a neutral non-polar amino acid, X₃ is serine or glycine, and X₈ is a neutral non-polar amino acid.

In yet another embodiment, the composition includes a polypeptide X₁-C-X₂-X₃-X₄-X₅-X₆-X₇-X₈-C-X₉, wherein C is cysteine, X₁, X₄, X₅, X₆, X₇, and X₉ are independently an amino acid residue, X₂ is a neutral non-polar amino acid, X₃ is a neutral polar amino acid, and X₈ is methionine or tryptophan.

In another embodiment, the composition includes a polypeptide X₁-C-X₂-X₃-X₄-X₅-X₆-X₇-X₈-C-X₉, wherein C is cysteine, X₁, X₄, X₅, X₆, X₇, and X₉ are independently an amino acid residue, X₂ is leucine or proline, X₃ is serine or glycine, and X₈ is methionine or tryptophan. In yet another embodiment, X₁, X₂, X₄, X₅, X₆, X₇, and X₉ are selected from naturally occurring amino acids.

In other embodiments, the polypeptide sequence has an amino-terminal acetyl group or a carboxy-terminal amide group. Furthermore, the polypeptide sequence can have both an amino-terminal acetyl group and a carboxy-terminal amide group. For example, when X₁ is an amino acid residue, it represents the N-terminus of the peptide, and it can be acetylated. Methodology for making terminal modifications as discussed herein are well known in the art (Fields (1997), Methods in Enzymology 289).

One skilled in the art will recognize that the proximity of the two cysteines in the amino acid sequence C-X₂-X₃-X₄-X₅-X₆-X₇-X₈-C results in the formation of a cyclic peptide due to the formation of a disulfide bond. This bond can be reduced as described in Example 6 or using other methods known in the art. Reduction and alkylation of sulfhydryl groups is well known in the art (see, e.g. Crestfield et al. (1963) J. Biol. Chem. 238:622-627). It is preferred that the peptide be oxidized (i.e. containing a disulfide bond) when using compositions described herein. Oxidation of peptides is well known in the art, and can be performed as described in, e.g., Fields (1997), Methods in Enzymology 289.

Compositions comprising any of the polypeptides described above can promote complement activation. The peptide sequences do so by binding to C5, and are independent of the complement pathway, i.e., they can activate both classical and alternative pathways. Exemplary peptides of the present invention, wherein X₁ and X₉ are each a hydrogen atom, include SEQ ID NO 1(peptide #1), SEQ ID NO 2 (peptide #2), and SEQ ID NO 3 (peptide #3).

The peptides with the above-mentioned consensus sequence were identified by panning a phage display library. Accordingly, a phage display library is a useful method for identifying peptides described herein. Phage display is well known in the art and describes a selection technique in which a peptide or protein is expressed as a fusion with a coat protein of a bacteriophage, resulting in display of the fused protein on the exterior surface of the phage virion, while the DNA encoding the fusion resides within the virion. Phage display can be used to create a physical linkage between a vast library of random peptide sequences to the DNA encoding each sequence, allowing rapid identification of peptide ligands for a variety of target molecules (antibodies, enzymes, cell-surface receptors, etc.) by an in vitro selection process called “panning.” In its simplest form, panning is carried out by incubating a library of phage-displayed peptides with a plate (or bead) coated with the target, washing away the unbound phage, and eluting the specifically-bound phage. Alternatively the phage can be reacted with the target in solution, followed by affinity capture of the phage-target complexes onto a plate or bead that specifically binds the target. The eluted phage is then amplified and taken through additional cycles of panning and amplification to successively enrich the pool of phage in favor of the tightest binding sequences. After several rounds, individual clones are characterized by DNA sequencing and ELISA.

For the purposes of the present invention, screening of the phage library against C5 can be performed by several different methods which include, but are not limited to: directly coating a surface with the target protein and then screening with the library; screening against biotinylated C5 immobilized on a neutravidin coated surface; or screening against C5a, the small fragment after proteolysis of C5 to determine if a site may be available on that fragment which is also present in C5. One of many possible methodologies suitable for phage display identification of complement inhibitors is detailed in Example 1. Modification of phage libraries or the panning protocol as described in Example 1 are well within the knowledge of a skilled artisan.

Within the scope of the present invention are polypeptide analogs of the invention arrived at by amino acid substitutions. One factor that can be considered in making amino acid substitutions is the hydropathic index of amino acids. The importance of the hydropathic amino acid index in conferring interactive biological function on a protein has been discussed by Kyte and Doolittle (J. Mol. Biol., 157: 105-132, 1982). It is accepted that the relative hydropathic character of amino acids contributes to the secondary structure of the resultant protein. This, in turn, affects the interaction of the protein with molecules such as enzymes, substrates, receptors, DNA, antibodies, antigens, etc.

Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant protein having similar biological activity, i.e., still retaining correct biological function. In making such changes, amino acids having hydropathic indices within ±2 are preferably substituted for one another, those within ±1 are more preferred, and those within ±0.5 are most preferred.

Furthermore, amino acid substitutions in the peptides of the present invention can be based on factors other than hydrophobicity, such as size, side chain substituents, charge, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. It should be noted that changes which are not expected to be advantageous can also be useful if these result in the production of functional sequences.

It will be appreciated by those of skill in the art that a peptide mimic may serve equally well as a peptide for the purpose of providing the specific backbone conformation and side chain functionalities required for binding to C5 and promoting complement activation. Accordingly, it is contemplated as being within the scope of the present invention to produce C5-binding, complement activation-promoting compounds through the use of either naturally-occurring amino acids, amino acid derivatives, analogs or non-amino acid molecules capable of being joined to form the appropriate backbone conformation. A non-peptide analog, or an analog comprising peptide and non-peptide components, is sometimes referred to herein as a “mimetic” or “peptidomimetic,” to designate substitutions or derivations of the peptides of the invention, which possess the same backbone conformational features and/or other functionalities, so as to be sufficiently similar to the exemplified peptides to augment complement activation. The use of peptidomimetics for the development of high-affinity peptide analogs is well known in the art (see, e.g., Zhao et al. (1995), Nature Structural Biology 2: 1131-1137; Beely, N. (1994), Trends in Biotechnology 12: 213-216; Hruby, V. J. (1993), Biopolymers 33: 1073-1082).

Skilled artisans will recognize that the amino acid sequences of the present invention and fragments, variants and analogs thereof can be synthesized by a number of different methods. All of the amino acid compounds of the invention can be made by chemical methods well known in the art, including, e.g., solid phase peptide synthesis and recombinant methods. Both methods are well known in the art.

The principles of solid phase chemical synthesis of polypeptides may be found in general texts in the area. See, e.g., H. Dugas and C. Penney, BIOORGANIC CHEMISTRY, (1981) Springer-Verlag, New York, pgs. 54-92. For example, peptides may be synthesized by solid-phase methodology utilizing an Applied Biosystems 430A peptide synthesizer (commercially available from Applied Biosystems, Foster City Calif.) and synthesis cycles supplied by Applied Biosystems. Protected amino acids, such as t-butoxycarbonyl-protected amino acids, and other reagents are commercially available from many chemical supply houses.

In another embodiment, the peptides of the present invention can be produced by classical solution peptide synthesis, also known as liquid-phase peptide synthesis. Polypeptides are also available commercially from, e.g., Sigma Chemical Co. (St. Louis, Mo.), Bachem Bioscience, Inc. (King Of Prussia, Pa.), and Peptides International (Louisville, Ky.).

In addition, the DNA sequences encoding the peptides or fragments, analogs or variants thereof can be produced by synthetic methods. The synthesis of nucleic acids is well known in the art. See, e.g., E. L. Brown, R. Belagaje, M. J. Ryan, and H. G. Khorana, Methods in Enzymology, 68:109-151 (1979). The DNA segments corresponding to the amino acid sequences described herein can be generated using conventional DNA synthesizing apparatus such as the Applied Biosystems Model 380A or 380B DNA synthesizers (commercially available from Applied Biosystems, Inc., 850 Lincoln Center Drive, Foster City, Calif. 94404) which employ phosphoramidite chemistry. In the alternative, the more traditional phosphotriester chemistry may be employed to synthesize the nucleic acids of this invention. See, e.g., OLIGONUCLEOTIDE SYNTHESIS, A PRACTICAL APPROACH, (M. J. Gait, ed., 1984).

Following the synthesis of DNA sequences, such sequences are produced by utilizing recombinant systems. The basic steps in the recombinant production of desired peptides are: a) construction of a synthetic or semi-synthetic DNA encoding the peptide of interest; b) integrating said DNA into an expression vector in a manner suitable for the expression of the peptide of interest, either alone or as a fusion protein; c) transforming an appropriate eukaryotic or prokaryotic host cell with said expression vector, d) culturing said transformed or transfected host cell in a manner to express the peptide of interest; and e) recovering and purifying the recombinantly produced peptide of interest.

The methods of recombinantly producing peptides/proteins are well known in the art. Literature that describes these techniques includes, for example, Sambrook, et al., Molecular Cloning: A Laboratory Manual, Cold Spring Harbor Laboratory, Cold Spring Harbor, N.Y. (2nd edition, 1989); Ausubel, et al., Current Protocols in Molecular Biology (1987); O'Reilly, et al., Baculovirus Expression Vectors: A Laboratory Manual (1992); Practical Molecular Virology (Collins, ed., 1991); Culture of Animal Cells: A Manual of Basic Technique (Freshney, ed., 2nd edition, 1989); J. Miller, Experiments in Molecular Genetics, Cold Spring Harbor Laboratory, Cold Spring Harbor, N.Y. (1972); D. A. Morrison, Transformation and Preservation of Competent Bacterial Cells by Freezing, Methods Enzymol. 68:326-331 (1979); and J. Perbal, A Practical Guide to Molecular Cloning, John Wiley & Sons (1984).

After the desired peptide is obtained either by chemical synthesis or recombinant methods, it can be isolated and purified using a number of procedures that are well known in the art, such as, e.g., extraction, precipitation, chromatography, affinity chromatography, electrophoresis, or the like.

The pharmaceutical compositions of the invention comprise a composition of the invention as an active ingredient in admixture with one or more pharmaceutically-acceptable vehicles and, optionally, with one or more other compounds, drugs, or other materials. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like). Suitable formulations and additional carriers are described in Remington's Pharmaceutical Sciences, (17.sup.th Ed., Mack Pub. Co., Easton, Pa.).

Pharmaceutically acceptable cations include metallic ions and organic ions. Metallic ions include, but are not limited to, appropriate alkali metal salts, alkaline earth metal salts and other physiologically acceptable metal ions. Exemplary ions include aluminum, calcium, lithium, magnesium, potassium, sodium and zinc in their usual valences. Organic ions include, but are not limited to, protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Pharmaceutically acceptable acids include without limitation hydrochloric acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid, oxalacetic acid, fumaric acid, propionic acid, aspartic acid, glutamic acid, benzoic acid, and the like.

Pharmaceutical preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation.

Pharmaceutical compositions of the invention suitable for administrations comprise one or more compositions of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient, or suspending or thickening agents.

Examples of suitable aqueous and nonaqueous vehicles which may be employed include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption, such as aluminum monosterate and gelatin, or by dissolving or suspending the composition(s) in an oil vehicle.

The formulations may be presented in unit-dose or multi-dose sealed containers (for example, ampoules and vials). The formulations may be stored in a lyophilized condition requiring only the addition of the sterile liquid carrier, for example water for injection, immediately prior to use.

In certain embodiments, the pharmaceutical compositions described herein can be provided as prodrugs. Prodrugs can be created, e.g., through the creation of a labile and reversible ester bond. By way of example, esterification of any of the X₁-X₉ can be used to create prodrugs. To achieve such esterification, the position is selected among X₁-X₉ based on the presence of an amino acid that contains either an alcohol or acid (carboxyl) group. For example, when using a natural amino acid at any of these positions, which contains an alcohol group (such as serine, threonine, tyrosine or hydroxyproline or hydroxylysine, these amino acids can be modified with an acid (such as acetic acid) to create an ester. Conversely, aspartic acid and glutamic acid as well as the carboxyl terminus can be esterified with alcohols such as ethanol to make esters. Non-proteogenic or non-natural/synthetic amino acids that contain either an alcohol or carboxylic acid group can also be modified in this manner. Upon administration to a patient, the prodrugs created in this way are converted to active compounds upon either the hydrolysis of the ester bond by esterases or by the action of the acid in the stomach.

It is another aspect of the present invention to provide a wound dressing comprising one or more of the pharmaceutical composition as described herein. Wound healing involves a complex series of interactions between many cell types and between cells and their extracellular matrix (ECM). Many cell types, cytokines, coagulation factors, growth factors and complement activation and matrix proteins, such as fibronectin and collagen contribute to healing in various proportions. The functions and precise mechanisms of the cellular, humoral and local factors are unclear and poorly understood.

By way of example, it is known that wound dressings comprising collagen can have a positive therapeutic effect on wound healing. It has been shown that collagen is chemotactic towards a variety of cell types, including neutrophils, monocytes, and fibroblasts. The chemotaxis is thought to be advantageous for wound healing. Furtermore, another important factor in wound healing is keeping the wound dressing and the wound as clean as possible to avoid infection. It is known that the complement system, and particularly the alternative and lectin pathway play a role in the immune surveillance, i.e. surveillance for the presence of any pathogens in the body. While not being bound to a particular theory, it is believed that the promotion of complement activation in a patient who is afflicted with a wound may be beneficial in warding off wound infections. Accordingly, the addition of the pharmaceutical compositions described herein to wound dressings is thought to be useful for preventing or reducing infections in patients suffering from wounds, who are in need of wound dressings. Furthermore, a number of bacteria and viruses have evolved mechanisms to evade the complement. Thus, augmentation of complement activation by said pharmaceutical compositions may be further beneficial for wound repair.

In certain aspects, the wound treatment composition according to the present invention is a liquid, gel or semi-solid ointment for topical application to a wound comprising the one or more peptides in a pharmaceutically acceptable carrier. Suitable carriers include: hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also including creams/ointments used for topical pharmaceutical preparations, e.g. creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, ad stabilisers.

In other aspects, the wound treatment composition is coated onto, or incorporated into a solid wound dressing such as a film, a fibrous pad or a sponge. The solid dressing may also be bioabsorbable. The pharmaceutical compositions can be simply coated onto the solid dressing by dipping, or may be covalently bound to, or may be dispersed therein as a solid solution. Suitable solid wound dressings include, e.g., the absorbent polyurethane foam available under the Registered Trade Mark TIELLE (Johnson & Johnson Medical, Inc.), fibrous alginate pads such as those available under the Registered Trade Mark KALTOSTAT (Convatec Corporation), and bioabsorbable collagen/alginate materials available under the Registered Trade Mark FIBRACOL (Johnson & Johnson Medical, Inc.).

In another embodiment, the pharmaceutical compositions of the present invention can be delivered as part of fibrin-based preparations. See, e.g., Wong et al., Thromb Haemos, 89:573-582, 2003. Briefly, biomatrix preparations such as fibrin-based biomaterials can act as provisional growth matrices for cells during wound repair of tissue-specific cellular and extracellular structures. The use of these fibrin-based biomaterials can be enhanced by adding specific bioactive agents in these biomaterials that can promote, e.g., cell growth, migration, etc. For wound repair, it is important to reduce chances of infection. Accordingly, the pharmaceutical compositions described herein can be used to enhance wound healing by including them in the fibrin-based biomaterials. Preparation of these modified biomaterials can be performed as generally described in Wong et al., discussed above.

It is another aspect of the present invention to provide an anti-tumor cell antigen antibody formulation which contains one or more of the pharmaceutical compositions described herein. Anti-tumor cell antigen antibody refers broadly to polyclonal and monoclonal IgG, IgM, IgA, IgD and IgE antibodies that are specific for tumor cells antigens. Generally, IgG and/or IgM are preferred because they are 1) the most common antibodies in the physiological situation, 2) activate complement, and 3) are most easily made in a laboratory setting. “Anti-tumor cell” antibody and “anti-tumor cell antigen” antibody are used herein interchangeably.

Polyclonal anti-tumor cell antibodies, obtained from antisera, may be employed in the invention. However, the use of monoclonal antibodies (MAbs) is generally preferred. MAbs are recognized to have certain advantages, e.g., reproducibility and large-scale production, that makes them suitable for clinical treatment. In one embodiment, the invention provides monoclonal antibodies of the murine, human, monkey, rat, hamster, rabbit and chicken origin. In another embodiment, an antibody is a monoclonal antibody, preferably of human or humanized mouse origin.

Humanized antibodies are generally chimeric monoclonal antibodies from mouse, rat, or other non-human species, bearing human constant and/or variable region domains (“part-human chimeric antibodies”). Mostly, humanized monoclonal antibodies for use in the present invention will be chimeric antibodies wherein at least a first antigen binding region, or complementarity determining region (CDR), of a mouse, rat or other non-human anti-tumor cell antigen monoclonal antibody is operatively attached to, or “grafted” onto, a human antibody constant region or “framework”. Humanized monoclonal antibodies for use herein may also be anti-tumor cell monoclonal antibodies from non-human species wherein one or more selected amino acids have been exchanged for amino acids more commonly observed in human antibodies. This can be readily achieved through the use of routine recombinant technology, particularly site-specific mutagenesis.

There are multiple ways to produce antibodies specific for tumor cell antigens. By way of example, the antibody-producing cells may be produced by fusing an anti tumor antibody producing cell with an immortal cell to prepare a hybridoma that produces such antibody.

Hybridoma-based monoclonal antibody preparative methods generally include the following steps:
(a) immunizing an animal with at least one dose, and optionally more than one dose, of an immunogenically effective amount of a tumor;
(b) preparing a collection of monoclonal antibody-producing hybridomas from the immunized animal;
(c) selecting from the collection at least one hybridoma that produces a monoclonal antibody;
(d) culturing the hybridoma to produce the antibody; and, (e) obtaining the monoclonal antibody from the cultured hybridoma.

As non-human animals are used for immunization, the anti-tumor cell antigen monoclonal antibodies obtained from such hybridomas will often have a non-human make up. Such antibodies may be optionally subjected to a humanization process, grafting or mutation, as known to those of skill in the art and further disclosed herein. Alternatively, transgenic animals, such as mice, may be used that comprise a human antibody gene library. Immunization of such animals will therefore directly result in the generation of human anti-tumor cell antibodies.

After the production of a suitable antibody-producing cell, most preferably a hybridoma, whether producing human or non-human antibodies, the monoclonal antibody-encoding nucleic acids may be cloned to prepare a “recombinant” monoclonal antibody. Any recombinant cloning technique may be utilized, including the use of PCR to prime the synthesis of the antibody-encoding nucleic acid sequences. Other powerful recombinant techniques are available that are ideally suited to the preparation of recombinant monoclonal antibodies. Such recombinant techniques include the phagemid library-based monoclonal antibody preparative methods comprising:
(a) immunizing an animal with at least one dose of tumor cells;
(b) preparing a combinatorial immunoglobulin phagemid library expressing RNA isolated from the antibody-producing cells, preferably from the spleen, of the immunized animal;
(c) selecting from the phagemid library a clone that expresses an anti-tumor specific antibody; and
(d) obtaining the nucleic acid sequence of the antibody.

Again, in such phagemid library-based techniques, transgenic animals bearing human antibody gene libraries may be employed, thus yielding recombinant human monoclonal antibodies.

Anti-tumor cell antibodies that may be formulated with pharmaceutical compositions described herein include but are not limited to: Campath, which is used to treat chronic lymphocytic leukemia, and Herceptin, used to treat breast cancer. While not being bound to a particular theory, the complement-based mechanisms by which the present invention may operate include complement-mediated lysis and complement-activated ADCC. “Complement-activated ADCC” is used to refer to the process by which complement, not an antibody Fc portion per se, holds a multi-component complex together and in which cells such as neutrophils lyse the target cell. For role of complement in tumor progression and treatment, see, e.g., Hakulinen J. Meri S., American Journal of Pathology. 153(3):845-55, Sep. 1998; Jarvis et al., International Journal of Cancer. 71(6):1049-55, Jun. 11, 1997; Bjorge et al., British Journal of Cancer. 75(9):1247-55, 1997; and Maenpaa et al., American Journal of Pathology. 148(4):1139-52, April 1996.

By way of example, rituximab (RITUXAN) is a genetically engineered murine/human monoclonal antibody directed against the CD20 antigen found on the surface of normal and malignant B lymphocytes. The antibody is an IgG1 immunoglobulin containing murine light- and heavy-chain variable region sequences and human constant region sequences. Rituximab is produced by in mammalian cell (CHO cell) suspension culture. The antibody is then purified by affinity and ion exchange chromatography. The purification process further includes specific viral inactivation and removal. Rituximab is stored as a preservative-free, liquid concentrate for intravenous (IV) administration. There are reports that complement activation is important for the therapeutic activity of rituximab. See, e.g., Di Gaetano et al. (J. Immunol., 171(3):1581-1587, Aug. 1, 2003).

Accordingly, the pharmaceutical compositions described herein may be formulated with anti-tumor cell antibodies, such as rituximab, in order to improve the therapeutic activity of such antibodies. The pharmaceutical compositions capable of promoting complement activation have been described in detail above. By way of example, a pharmaceutical composition containing the peptide of SEQ ID NO 1 may be formulated with, e.g., rituximab. Furthermore, the pharmaceutical compositions may be formulated as part of the liquid concentrate in which an anti-tumor cell antibody, such as, e.g., rituximab is formulated. By way of example, rituximab is formulated at a concentration of 10 mg/ml in 9.0 mg/ml sodium chloride, 7.35 mg/ml sodium citrate dihydrate, 0.7 mg/ml polysorbate 80, and sterile water, with pH adjusted to 6.5. Accordingly, the pharmaceutical compositions(s) described herein may be added to such antibody formulation in an amount effective to stimulate complement activation. The pharmaceutical compositions are previously prepared in such a way to be compatible with antibody formulation (in terms of pH, salt concentrations, added preservatives, etc.). Such considerations are well within the knowledge of one of ordinary skill in the art.

In addition to anti-tumor cell antigen antibodies, the pharmaceutical compositions described herein can be used with other antibody formulations, whose function may be enhanced by complement activation.

In another embodiment, the pharmaceutical compositions described herein may be administered to a patient undergoing a surgery. In a preferred embodiment, a surgery is a gastrointestinal surgery, due to a high risk of infection. The pharmaceutical compositions may be administered prior to the surgery, at the time of surgery, or post-surgery. A skilled artisan can readily determine the appropriate time window, depending on the health of the patient, type of surgery, chance of infection, etc. The pharmaceutical compositions that promote complement activation may be administered parenterally, for example intravenously, intramuscularly, subcutaneously, and the like.

The pharmaceutical compositions of the present invention may also be applicable in another clinical setting, where it is desirable to promote liver regeneration. The liver is one of the few mammalian organs that can replace damaged tissue following trauma, such as surgery, or after viral infections or chemically-induced toxic insults. Furthermore, the complement system is known to be involved in liver regeneration. See, e.g., Mastellos et al. et al. (J. immunol. 166:2479-2486, 2001), which shows that mice deficient in C5 display defective liver regeneration that can be at least partially restored with infusion of C5 or C5a, and Daveau et al. (J. immunol. 173:3418-3424, 2004), which shows that C5a receptor (C5aR) is up-regulated during liver regeneration and that binding of C5a to C5aR promotes a growth response. Also, Markiewski et al. (J. immunol. 173:747-754, 2004) demonstrated that CCl₄-induced liver damage activates C3 in mice. Thus, while not being bound to a theory, it is believed that the present pharmaceutical compositions may be useful in augmenting liver regeneration in patients with liver damage.

Effective dosage forms, modes of administration and dosage amounts of the composition of the invention may be determined empirically, and making such determinations is within the skill of the art. It is understood by those skilled in the art that the dosage amount will vary with the particular composition employed, the condition being treated, the severity of the condition, the route of administration, the rate of excretion, the duration of the treatment, the identity of any other drugs being administered to the mammal, the age, size and species of the mammal, and like factors well known in the medical and veterinary arts. In general, a suitable daily dose of a compound of the present invention will be that amount which is the lowest dose effective to produce a therapeutic effect. However, the total daily dose will be determined by an attending physician or veterinarian within the scope of sound medical judgment. If desired, the daily dose may be administered in multiple sub-doses, administered separately at appropriate intervals throughout the day.

When administered to treat a patient, who may benefit from complement activation, the pharmaceutical compositions may be administered in an amount effective to promote bacteriocidal activity of the complement. While not being bound to a particular theory, it is believed that this effect is achieved through lysis of the bacterial cell via MAC complex, and action of phagocytic cells that migrate in response to C5a.

When administered as part of a wound dressing, the pharmaceutical compositions are administered in an amount effective to promote increased immuno-surveillance by the complement system, which results in increased bacteriocidal action if bacteria are present.

When administered as part of an anti-tumor cell antibody formulation, the pharmaceutical compositions described herein are administered in an amount effective to promote complement-mediated tumor cytotoxicity or complement-dependent ADCC of tumor cells. A skilled artisan can readily determine therapeutically effective amount of said pharmaceutical compositions for any of the above-listed applications.

For therapeutic purposes, formulations for parenteral administration can be in the form of aqueous or non-aqueous isotonic sterile injection solutions or suspensions. These solutions and suspensions can be prepared from sterile powders or granules having one or more of the carriers or diluents. Injectable preparations, for example, sterile injectable aqueous or oleaginous suspensions, can be formulated according to the known art using suitable dispersing or wetting agents and suspending agents. The sterile injectable preparation may also be a sterile injectable solution or suspension in a nontoxic parenterally acceptable diluent or solvent. Among the acceptable vehicles and solvents that may be employed are water, Ringer's solution, and isotonic sodium chloride solution. In addition, sterile, fixed oils are conventionally employed as a solvent or suspending medium. For this purpose, any bland fixed oil may be employed, including synthetic mono- or diglycerides. In addition, fatty acids such as oleic acid are useful in the preparation of injectables. Dimethyl acetamide, surfactants including ionic and non-ionic detergents, and polyethylene glycols can be used. Mixtures of solvents and wetting agents such as those discussed above are also useful. Other adjuvants and modes of administration are well and widely known in the pharmaceutical art. For example, administration of the pharmaceutical compositions may be via the pulmonary route.

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

All publications and patent applications cited in this specification are herein incorporated by reference as if each individual publication or patent application was specifically and individually indicated to be incorporated by reference.

The following examples illustrate the invention, but are not to be taken as limiting the various aspects of the invention so illustrated.

### EXAMPLES

### Example 1

### Peptide Identification

Peptides were identified by panning the phage library PHD C7C (New England Biolabs) against complement component C5. The phage library was panned against C5 using four different methods for immobilizing C5. The binding reactions all contained 2×10¹¹ phage particles in PBS containing 0.5% BSA and 0.3% Tween 20. The binding reactions were carried out at 25° C. for two hours and then unbound phage were removed by washing 5 times with PBS containing 0.5% BSA and 0.3% Tween20. In the first three screening methods the phage were eluted by acid (0.2 M Glycine pH 2.2, 1 mg/mL BSA) and immediately neutralized with 1 M Tris pH 9.1. Following elution, recovered phage were amplified in ER2738 E. coli (NEB) and subjected to two more rounds of panning as described above. The methods for immobilizing C5 included directly coating C5 (Advanced Research Technologies) to the surface (1 ug/mL), capturing biotinylated C5 (1 ug/mL) on a neutravidin (Pierce) coated surface, and carrying out binding of phage with biotinylated C5 (500 ng) in solution followed by capture of the C5-phage complex on a neutravidin coated surface. The final method for panning was carried out in a manner similar to that previously described (e.g., Balass et al., Analytical Biochemistry 243(2):264-9, 1996) for the potential of identifying high affinity clones. Nitro-tyrosyl neutravidin (Balass et al., Analytical Biochemistry 243(2):264-9, 1996) was prepared as previously described and then coated on the surface of a plate. Unmodified biotin binding pockets were blocked by incubating the nitro-tyrosyl neutravidin with 0.6 mM free biotin in PBS for 30 minutes and then washed with 50 mM sodium carbonate buffer pH 10 for 30 minutes. Biotinylated C5 was bound to the nitro-tyrosyl neutravidin and then incubated with the phage as described above. Phage were eluted by first washing with acid and discarding to remove the low affinity clones and then eluting with 0.1 mg/mL of free biotin in 50 mM sodium carbonate buffer for 20 minutes. For the three screening methods which used neutravidin or nitro-tyrosyl neutravidin, a negative selection was performed in the final round against the corresponding neutravidin. DNA from twenty randomly selected clones from each of the four final libraries was isolated and sequenced.

Twenty clones were sequenced from the final round of panning from each of the four methods. One peptide sequence was isolated from all four methods of panning and two other peptide sequences were isolated from three of the four panning methods. These three peptides (peptide 1-CLSAHHHMC; peptide 2-CPSSPPHMC; and peptide 3-CPGKASPWC) accounted for 88% of all of the clones sequenced, indicating they had been amplified through rounds of panning and may be specific for binding to C5.

Synthetic free peptides (peptide 1, peptide 2, and peptide 3) of the three clones identified via panning were made. The peptides were synthesized by Alpha Diagnostic (San Antonio, Tex.). A tyrosine was added as the N terminal residue to each peptide and three glycines were added to the C terminus of each peptide to mimic the linker between the peptide and pill protein in the phage library and the disulfide bonds were oxidized.

### Example 2

### Phage clone binding to C5

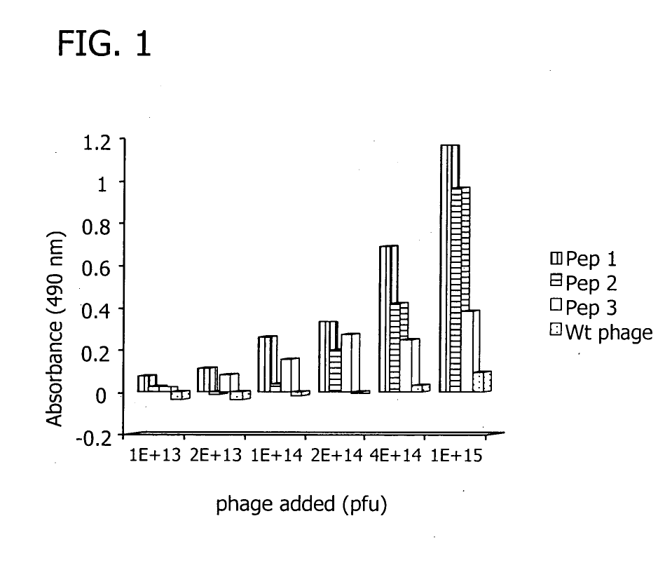

To confirm that Peptides 1, 2, and 3 (Sequence I. D. Nos:1, 2, and 3, respectively) bind C5, the phage clones were tested in a binding assay with increasing amounts of phage incubated with constant biotinylated C5, captured on a neutravidin plate and detected with an anti-phage antibody. Increasing concentrations of each phage clone were incubated for 2 hours with 200 ng of biotinylated C5 in PBS containing 0.5% BSA. The phage bound C5 complex was captured on a neutravidin coated microplate for 20 minutes at room temperature and then washed well. A peroxidase labeled anti-M13 antibody (Pharmacia) was used to detect the amount of phage bound to C5 with OPD substrate. Results showed that all three clones demonstrated significant binding to C5 compared to the wild type phage, with Peptide 1 having the tightest relative affinity, followed by Peptide 2, then Peptide 3 (FIG. 1).

### Example 3

### Hemolytic Assay

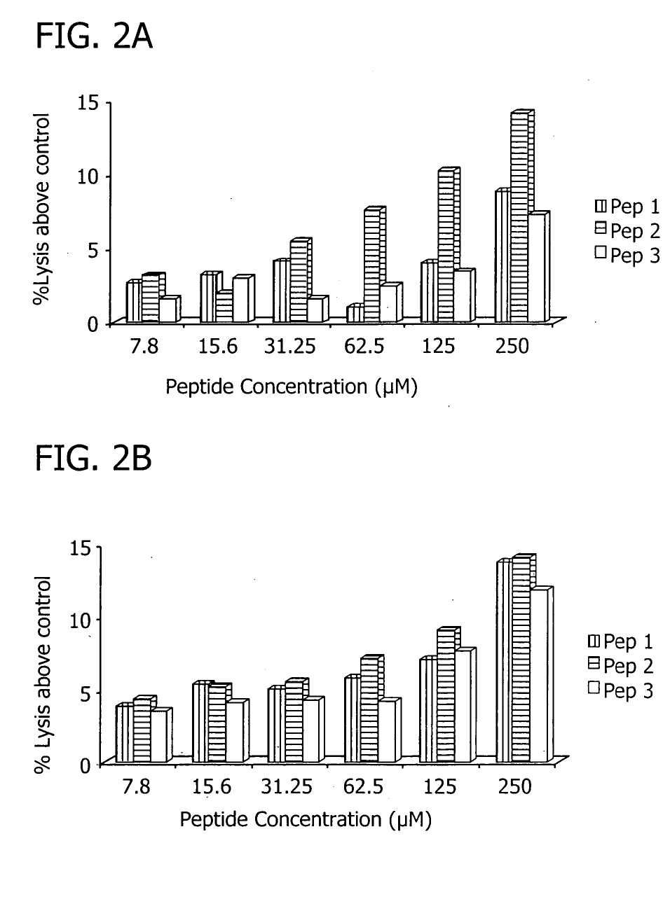

Activation of the classical and alternative pathway by the peptides was measured using standard hemolysis assays. For the classical pathway various concentrations of peptide were incubated with 50 ul pooled human plasma (diluted 1:30 with GVB++), 5×10⁷ antibody sensitized sheep erythrocyte cells, and GVB++buffer to a final volume of 200 ul. The reaction was incubated at 37° C. for one hour and centrifuged. The percentage of lysis was determined by measuring the optical density of the supernatant at 414 nm. Results indicated that all three of the peptides increased lysis of the erythrocytes in a concentration dependent manner (FIG. 2a). Lysis of the erythrocytes was not observed when the peptides were incubated in the absences of a source of complement (plasma). The peptides altered the activity of the complement protein(s) and increased the level of activation of the complement cascade resulting in an increase in hemolysis. Furthermore, when the disulfide bond of peptide 2 was reduced and the cysteines alkylated, enhancement of erythrocyte lysis was reduced (data not shown).

The peptides were also tested in an alternative assay using rabbit erythrocytes. In the alternative pathway assay, various concentrations of peptide were incubated with 50 ul of pooled human plasma (diluted 1:10 with GVB/MgEGTA), 2.5×10⁷ rabbit erythrocytes, 8 mM EGTA, 2 mM MgCl2, and GVB up to a final volume of 150 ul. The reaction mixture was incubated for 1 hour at 37° C., centrifuged, and the optical density at 414 nm of the supernatant was determined. Results with rabbit erythrocytes were similar to those in sheep erythrocytes (FIG. 2b). The results correlated with the peptides binding to C5 and causing increased activity of the classical and alternative pathway C5 convertases on cleavage of C5.

### Example 4

### Measurement of C5 Activation

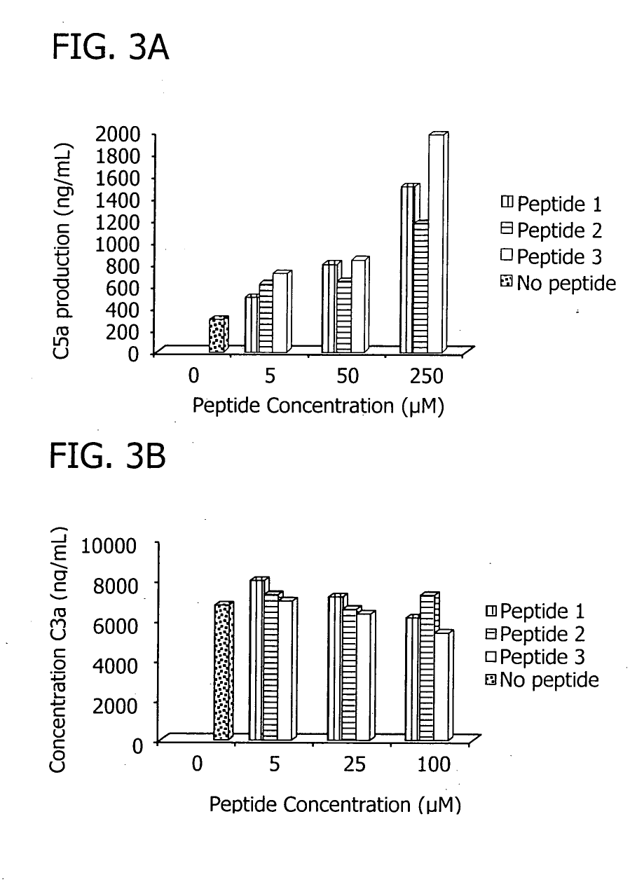

To confirm that the peptides caused an increase in complement activity at the C5 convertase step of the cascade, C3a and C5a production in complement activated plasma was examined in the presence of the Peptides 1-3. Specific activation of C5 convertase activity by the peptides in pooled human plasma was measured as follows. Varying concentrations of peptide were incubated with zymosan ranging in concentrations from 0.5 to 10 mg/mL in undiluted pooled human plasma. The reactions were incubated at 37° C. for one hour and then stopped by the addition of EDTA to a concentration of 5 mM. The samples were centrifuged and the plasma removed and analyzed for the levels of C3a and C5a. Concentrations of each of these complement components were determined by ELISAs using commercially available kits for C3a (Quidel) and C5a (BD Biosciences). Results showed that all three peptides caused an increase in C5a levels but relatively constant C3a levels in the presence of increasing peptide concentrations when complement is activated by zymosan (FIGS. 3a and 3b). Thus the peptides were selective for activating C5 convertase activity while not affecting C3 convertase activity. To determine whether the effect of the peptides were additive, all three peptides were added to the assay at 25 uM each and the level of C5a production was determined. With all three peptides present C5a was produced at 646 ng/mL. This level is comparable to the level of C5a production with just one peptide present at that concentration and does not appear to be an additive effect of the three peptides. Although the three peptides differ substantially in sequence, they may all be binding C5 at approximately the same site.

### Example 5

### Bactericidal Assay

The ability of Peptides 1- 3 to enhance complement-mediated killing of bacteria was examined. A virulent strain of E. coli that was chosen for the assays. E. coli strain 07:K1:NM (ATCC 23503), a strain less susceptible to complement mediated killing than most strains of E. coli, were grown in LB media to an optical density (OD) at 600 nm between 0.3 to 0.4 at 37° C. The cells were washed and resuspended in PBS containing 0.5% BSA and the concentration of cells determined from the OD at 600 nm. The cells were diluted in PBS/0.5% BSA to a concentration of 2×10⁵ cfu/mL and 100 ul of cells were added to 100 ul of pooled human plasma containing 100 uM of peptide. The sample was incubated for one hour at 37° C. 10 ul aliquots were removed before and after the incubation and bacterial survival was examined by the colony count method (counting cells on LB agar before and after incubation). Heat inactivated plasma (56° C., 30 min.), plasma with no peptide, and PBS/0.5% BSA were also incubated with the cells as controls.

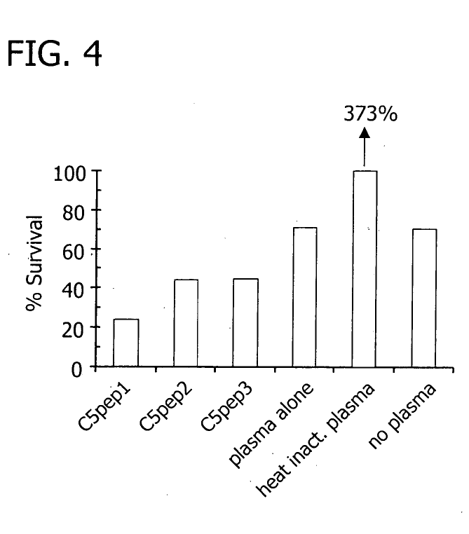

Results (see FIG. 4) indicated that Peptide 1 decreased bacterial survival from 71% for plasma alone to 23% with the addition of 100 uM Peptide 1. When the complement system was shut down by using heat-inactivated plasma, the bacteria proliferated well, resulting in 373% of the cells before incubation. Therefore, in the rich nutrient environment in the absence of complement, E. coli 07:K1:NM grew extremely well. Thus, it was necessary not only to kill the cells initially added to the assay, but also prevent further growth of the cells. The native complement system in plasma mediates some lysis of the bacteria, since the survival rate is 71% compared to the 373% survival in the absence of complement. However, the addition of the activating peptides (i.e., Peptides 1-3) further decreased the survival rate of the bacteria.

### Example 6

### Reduction of Peptide

Reduction and alkylation of the disulfide bond in the peptide was carried out by incubation peptide with a 10 fold molar excess of DTT in degassed 250 mM Tris pH 8.5 for 2 hours. The reaction was quenched by the addition of a 50 fold molar excess of iodoacetamide and allowed to react for an additional hour. The reduced and alkylated peptide was purified over an RP-8 column on a Waters 490 HPLC with a gradient of acetonitrile from 0-80%, containing 0.1% trifluoroacetic acid.

1. A composition for promoting complement activation, said composition comprising a polypeptide comprising the sequence X₁-C-X₂-X₃-X₄-X₅-X₆-X₇-X₈-C-X₉, wherein
C is cysteine;
X₁ is a hydrogen atom, an amino acid residue, or a bond covalently linking the polypeptide to another component of the composition;
X₂ is a neutral non-polar amino acid residue;
X₃ is a neutral polar amino acid residue;
X₄, X₅, X₆, and X₇ are independently any amino acid residue;
X₈ is a neutral non-polar amino acid residue; and
X₉ is a hydrogen atom, an amino acid residue, or a bond covalently linking the polypeptide to another component of the composition.

2. The composition of claim 1 wherein X₁ is an amino acid residue, and X₉ is an amino acid residue.

3. The composition of claim 1, wherein X₁ is a hydrogen atom.

4. The composition of claim 1, wherein X₉ is a hydrogen atom.

5. The composition of claim 1, wherein X₂ is leucine or proline.

6. The composition of claim 1, wherein X₃ is serine or glycine.

7. The composition of claim 1, wherein X₈ is methionine or tryptophan.

8. The composition of claim 1, wherein X₁ is a bond covalently linking the polypeptide of claim 1 to another component of the composition, wherein said component is selected from the group consisting of peptides, vitamins, carbohydrates, polysaccharides, lipids, lipopolysaccharides, nucleic acids, and biomaterials.

9. The composition of claim 8, wherein the component is a peptide.

10. The composition of claim 1, wherein X₉ is a bond covalently linking the polypeptide of claim 1 to another component of the composition, wherein said component is selected from the group consisting of peptides, vitamins, carbohydrates, polysaccharides, lipids, lipopolysaccharides, nucleic acids, and biomaterials.

11. The composition of claim 10, wherein the component is a peptide.

12. The composition of claim 1, wherein the two cysteine residues are linked with a disulfide bond.

13. The composition of claim 1, wherein the polypeptide is selected from the group consisting of SEQ ID NO 1, SEQ ID NO 2, and SEQ ID NO 3.

14. The composition of claim 13, wherein the polypeptide is SEQ ID NO 1.

15. The composition of claim 13, wherein the polypeptide is SEQ ID NO 2.

16. The composition of claim 13, wherein the polypeptide is SEQ ID NO 3.

17. A composition for promoting complement activation, said composition comprising a polypeptide comprising the sequence X₁-C-X₂-X₃-X₄-X₅-X₆-X₇-X₈-C-X₉ wherein:
C is cysteine;
X₁, X₄, X₅, X₆, X₇, and X₉ are independently an amino acid residue;
X₂ is leucine or proline;
X₃ is serine or glycine; and
X₈ is methionine or trypthophan.

18. A pharmaceutical composition for promoting complement activation, said composition comprising a polypeptide comprising the sequence X₁-C-X₂-X₃-X₄-X₅-X₆-X₇-X₈-C-X₉ and a pharmaceutically effective carrier or excipient, wherein
C is cysteine;
X₁ is a hydrogen atom, an amino acid residue, or a bond covalently linking the polypeptide to another component of the composition;
X₂ is a neutral non-polar amino acid residue;
X₃ is a neutral polar amino acid residue;
X₄, X₅, X₆, and X₇ are independently any amino acid residue;
X₈ is a neutral non-polar amino acid residue; and
X₉ is a hydrogen atom, an amino acid residue, or a bond covalently linking the polypeptide to another component of the composition.

19. The composition of claim 18, wherein X₁ is an amino acid residue, and X₉ is an amino acid residue.

20. The pharmaceutical composition of claim 18, wherein X₁ is a hydrogen atom.

21. The pharmaceutical composition of claim 18, wherein X₉ is a hydrogen atom.

22. The pharmaceutical composition of claim 19, wherein X₁ is modified with an acetyl group.

23. The pharmaceutical composition of claim 19, wherein X₉ is modified with an amide group.

24. The pharmaceutical composition of claim 18, wherein X₂ is leucine or proline.

25. The pharmaceutical composition of claim 18, wherein X₃ is serine or glycine.

26. The pharmaceutical composition of claim 18, wherein X₈ is methionine or tryptophan.

27. The pharmaceutical composition of claim 18, wherein the two cysteine residues are linked with a disulfide bond.

28. The pharmaceutical composition of claim 18, wherein X₁ is a bond covalently linking the polypeptide to another component of the composition, wherein said component is selected from the group consisting of peptides, vitamins, carbohydrates, polysaccharides, lipids, lipopolysaccharides, nucleic acids, and biomaterials.

29. The pharmaceutical composition of claim 28, wherein the component is a peptide.

30. The pharmaceutical composition of claim 18, wherein X₉ is a bond covalently linking the polypeptide to another component of the composition, wherein said component is selected from the group consisting of peptides, vitamins, carbohydrates, polysaccharides, lipids, lipopolysaccharides, nucleic acids, and biomaterials.

31. The pharmaceutical composition of claim 30, wherein the component is a peptide.

32. The pharmaceutical composition of claim 19, wherein
X₂ is leucine or proline;
X₃ is serine or glycine; and
X₈ is methionine or trypthophan.

33. The pharmaceutical composition of claim 18, wherein the polypeptide is selected from the group consisting of SEQ ID NO 1, SEQ ID NO 2, and SEQ ID NO 3.

34. The pharmaceutical composition of claim 33, wherein the polypeptide is SEQ ID NO 1.

35. The pharmaceutical composition of claim 33, wherein the polypeptide is SEQ ID NO 2.

36. The pharmaceutical composition of claim 33, wherein the polypeptide is SEQ ID NO 3.

37. A method for promoting complement activation in a patient by administering to the patient a therapeutically effective amount of the pharmaceutical composition of claim 18.

38. The method of claim 37, wherein said pharmaceutical composition comprises a polypeptide comprising the sequence X₁-C-X₂-X₃-X₄-X₅-X₆-X₇-X₈-C-X₉, wherein
X₁ is an amino acid residue;
X₂ is leucine or proline;
X₃ is serine or glycine;
X₈ is methionine or trypthophan; and
X₉ is an amino acid residue.

39. The method of claim 37, wherein the pharmaceutical composition is administered parenterally.

40. The method of claim 37, wherein the pharmaceutical composition comprises the polypeptide selected from the group consisting of SEQ ID NO 1, SEQ ID NO 2, and SEQ ID NO 3, or mixtures thereof.

41. A wound dressing comprising a therapeutically effective amount of the pharmaceutical composition of claim 18.

42. The wound dressing of claim 41, wherein the pharmaceutical composition comprises a polypeptide comprising the sequence X₁-C-X₂-X₃-X₄-X₅-X₆-X₇-X₈-C-X₉, wherein
X₁ is an amino acid residue;
X₂ is leucine or proline;
X₃ is serine or glycine;
X₈ is methionine or trypthophan; and
X₉ is an amino acid residue.

43. The wound dressing of claim 41, wherein the pharmaceutical composition comprises the polypeptide selected from the group consisting of SEQ ID NO 1, SEQ ID NO 2, and SEQ ID NO 3, or mixtures thereof.

44. An anti-tumor cell antigen antibody formulation, comprising a therapeutically effective amount of the pharmaceutical composition of claim 18.

45. The anti-tumor cell antigen antibody formulation of claim 44, wherein the pharmaceutical composition comprises the polypeptide selected from the group consisting of SEQ ID NO 1, SEQ ID NO 2, and SEQ ID NO 3, or mixtures thereof.
